(19) United States
(12) Patent Application Publication
(10) Pub. No.: US 2007/0286904 A1
(43) Pub. Date: Dec. 13, 2007

(54) STRUCTURED MATERIALS

(76) Inventors: Lewis Michael Popplewell, Morganville, NJ (US); Lulu Henson, Plainsboro, NJ (US)

Correspondence Address: INTERNATIONAL FLAVORS & FRAGRANCES INC., 521 WEST 57TH ST, NEW YORK, NY, 10019, US

(21) Appl. No.: 11/844,376

(22) Filed: Aug. 24, 2007

Related U.S. Application Data
(63) Continuation-in-part of application No. 11/216,695, filed on Aug. 31, 2005, now abandoned.

(51) Int. Cl. A61K 9/14; A23D 9/00; A23L 1/22; C11D 17/00; A61P 43/00; A61K 8/02

(52) U.S. Cl. 424/499; 426/534; 426/601; 510/108; 512/1

(57) ABSTRACT

The present invention relates to a structured material comprising: a hydrophilic particle such as a hydrophilic silica, a silicate, a dextrin, a starch, a mineral, sodium bicarbonate, a clay, a sugar, a polyol, a dried flavor/fragrance, a hydrocolloid, a protein, a cellulose, methylcellulose, hydroxypropyl methyl cellulose, a wheat flour, corn, potato, rice, a dried milk and dairy powder, a spice, a herb, a vegetable powder, a meat powder, a salt, and an acid; a hydrophobic active ingredient selected from the group consisting of a flavor, a fragrance, a pharmaceutical and nutritional agent, and/or an appropriate hydrophobic carrier such as a triglyceride oil, a triglyceride fat, and a mineral oil; and a polyhydric material such as glycerine, propylene glycol, dipropylene glycol, polyethylene glycol, aqueous solutions of sorbitol, isomalt, lactitol, and maltitol, an aqueous solution of a sugar, a starch, an acid, a polyethylene glycol, a dextrin, and a water soluble or swellable compound, or water, the hydrophobic active ingredient is 50-90% by weight of the structured material, and the ratio of the hydrophilic particle to the polyhydric material is about 0.5 and greater.

### FIELD OF THE INVENTION

The present invention relates to stable structured materials, their preparation, and their use in the flavor, fragrance, and pharmaceutical industries.

### BACKGROUND OF THE INVENTION

It is well known that liquid active ingredients such as flavors, fragrances, therapeutic or diagnostic agents and oils may be absorbed onto a variety of hydrophobic and hydrophilic solid particles in order to render them easier to handle and distribute. Often, fine particles with a large externally-available porosity are used due to their ability to carry high levels of these active ingredients while still retaining good flow properties. The porosity provides a large available surface area for absorption of the liquid film. Materials of this type include selected silicon dioxides, such as Syloid 244 by Grace Davison, tapioca dextrins such as N-Zorbit™ by National Starch and polypropylene, such as Accurel by Membrana GmbH. From the preceding examples, it is recognized that the surface of the particle may be either hydrophilic or hydrophobic, and that selection of the surface type will be dependent on the specific application.

The level of the absorbed active ingredient needs to be kept below a certain level in order to maintain the particle-active ingredient mixture as a flowable powder or granular product. This level is specific to each particle-active ingredient combination, and is normally determined experimentally. As the level of active ingredient is increased, the mixture becomes agglomerated and sticky. If the level is further increased, a paste, and finally a liquid suspension results. These suspensions are often useful, as they will have an increased viscosity in comparison to the original liquid which can make handling easier in specific situations.

U.S. Pat. No. 6,184,220 reveals a siloid structure that incorporates low levels of active ingredient, silicon dioxide, and hydrophilic polymer in an aqueous buffer system. To form such a siloid structure, water is required as there is essentially no direct interaction between the active ingredient and silicon dioxide in the absence of water. While an increase of silicon dioxide and/or hydrophilic polymer increase viscosity in the mixture, proper siloid structure can not be formed with a low or no amount of water. Further, simple dispersion (i.e., without an inter-molecule structure) of silicon dioxide particles in the mixture of active ingredient and hydrophilic polymer does not help significantly in controlling the delivery of the active ingredient such as a flavor or a fragrance, as the dispersion is not significantly stable in an aqueous application system most often encountered.

Accordingly, a need exists to develop stable structures that contain high levels of active ingredient and that allow a controlled method of delivery of the active ingredient.

### SUMMARY OF THE INVENTION

The present invention relates to a structured material comprising:
a hydrophilic particle such as a hydrophilic silica, a silicate, a dextrin, a starch, a mineral, sodium bicarbonate, a clay, a sugar, a polyol, a dried flavor/fragrance, a hydrocolloid, a protein, a cellulose, methylcellulose, hydroxypropyl methyl cellulose, a wheat flour, corn, potato, rice, a dried milk and dairy powder, a spice, a herb, a vegetable powder, a meat powder, a salt, and an acid;
a hydrophobic active ingredient selected from the group consisting of a flavor, a fragrance, a pharmaceutical and nutritional agent, and/or an appropriate hydrophobic carrier such as a triglyceride oil, a triglyceride fat, and a mineral oil; and
a polyhydric material such as glycerine, propylene glycol, dipropylene glycol, polyethylene glycol, aqueous solutions of sorbitol, isomalt, lactitol, and maltitol, an aqueous solution of a sugar, a starch, an acid, a polyethylene glycol, a dextrin, and a water soluble or swellable compound, or water,
the hydrophobic active ingredient is 50-90% by weight of the structured material, and the ratio of the hydrophilic particle to the polyhydric material is about 0.5 and greater.

The above composition of the present invention will be apparent by reading the following specification.

### DETAILED DESCRIPTION OF THE INVENTION

It has been unexpectedly found that active ingredients such as flavors, fragrances, and pharmaceutical, therapeutic and diagnostic agents may be effectively structured into stable solids, or viscous liquids, using hydrophilic particles and polyhydric alcohol or like materials of proper amounts. While the exact mechanism has not been proven, it is thought that the polyhydric alcohol creates an association between the particles, thus resulting in a more stable structure than what can be obtained with particles alone. This allows the structures formed to be used effectively in delivering active ingredients. Specifically, these structures have been found useful in delivering flavors and fragrances in basic two ways:
a) The product is a structured liquid flavor or fragrance. These structures have been found to have stability in aqueous applications such as toothpaste; and
b) The product is a structured fat or oil, which contains dried particles of flavor or fragrance. These structures provide improved hydrophobic protection to the flavor or fragrance in a variety of aqueous applications.

The above approaches may be combined by using dried flavor/fragrance inside a structured liquid flavor or fragrance product or diluting a liquid flavor or fragrance using a fat or oil.

The concept of the present invention is clearly illustrated by the experiments described in the examples below. As indicated in Example 3, an addition of about 5% of polyhydric alcohol glycerine approximately doubles the viscosity of the structured material. In this case, the viscosity measured using a Haake AR 2000 Rheometer and a shear rate sweep is from about 10,000 to about 140,000 Pa.s at about 25° C. The viscosity of the same material under the same conditions but without the polyhydric alcohol is in the range of from about 5,000 to about 55,000. The additional portion of the polyhydric alcohol increases the viscosity of the structured material. As further indicated in the same example, an addition of about 8% of polyhydric alcohol glycerine to a sample containing a higher level of hydrophilic particles also greatly increases the viscosity of the structured material. In this case the viscosity measured using a Haake AR 2000 Rheometer and a shear rate sweep is from about 80,000 to about 1,000,000 Pa.s at about 25° C. Further, as indicated in Example 4, it is not possible to create a structured monophasic or uniform product at a low silicon dioxide to glycerine ratio such as 0.17 or 0.10. The presence of liquid peppermint flavor or triglyceride as a separate phase in the mixture defeats the purpose of creating a delivery system that facilitates handling and with modified release characteristics compared to the flavor or triglyceride alone. Furthermore, as indicated in Example 5, the ratio of the peppermint flavor to Aerosil (Aerosil 200; fumed silicon dioxide) and glycerine must be carefully formulated. IFF Example 5 shows a range of possible compositions. Increasing the amounts of Aerosil and glycerine increases the viscosity as expected. However, no structured material is obtained unless the ratio of Aerosil to glycerine reaches about 0.5 and greater. Acceptable formulations are those that form a uniform or homogeneous viscous fluid or semi-solid or solid product.

The structured materials of the present invention are formulated as follows:

Hydrophobic Active Ingredient: 50-90%, preferably 55-75%, most preferably 60-70% by weight of the structured material;

Hydrophilic Particles: 5-70%, preferably 10-50%, most preferably 20-30% by weight of the structured material; and

Liquid polyhydric material: 1-20%, preferably 10- 15% by weight of the structured material,

whereby the ratio of the hydrophilic particles to the liquid polyhydric material: about 0.5 and greater, preferably about 1.0 and greater, more preferably about 1.2 and greater, most preferably about 1.4 and greater.

As used herein the term structured material is understood to mean a material exhibiting increased viscosity, resistance to deformation, or the properties of a solid; liquid polyhydric material is any material with more than one —OH group, water, or an aqueous solution of a soluble material; hydrophobic material is any material that tends not to dissolve or mix with water.

The active ingredient provided by the present invention consists largely of hydrophobic materials, although it may contain materials with some aqueous solubility, or materials with high water solubility in limited amounts. It may be any suitable agent including therapeutic and diagnostic agents, flavors, fragrances, triglyceride oils/fats, mineral oils and combinations thereof. Emulsifiers/surfactants such as mono-glycerides, di-glycerides, and polysorbates may form up to 30% of the structured material as well. The material must be a fluid during mixing.

The hydrophobic carrier material such as triglyceride oil may also solubilize the flavor or fragrance hydrophobic active ingredient and serve as the base for the hydrophobic particle. The amount of flavor/fragrance that is dissolved in the solvent may vary depending on the flavor/fragrance impact desired in the product.

A list of suitable fragrances is provided in U.S. Pat. No. 4,534,891. Another source of suitable fragrances is found in Perfumes, Cosmetics and Soaps, Second Edition, edited by W. A. Poucher, 1959. Among the fragrances provided in this treatise are acacia, cassie, chypre, cyclamen, fern, gardenia, hawthorn, heliotrope, honeysuckle, hyacinth, jasmine, lilac, lily, magnolia, mimosa, narcissus, freshly-cut hay, orange blossom, orchid, reseda, sweet pea, trefle, tuberose, vanilla, violet, wallflower, and the like.

Conventional flavoring materials useful in flavoring products include saturated fatty acids, unsaturated fatty acids and amino acids; alcohols including primary and secondary alcohols, esters, carbonyl compounds including ketones, other than the dienalkylamides of our invention and aldehydes; lactones; other cyclic organic materials including benzene derivatives, acyclic compounds, heterocyclics such as furans, pyridines, pyrazines and the like; sulfur-containing compounds including thiols, sulfides, disulfides and the like; proteins; lipids, carbohydrates; so-called flavor potentiators such as monosodium glutamate; magnesium glutamate, calcium glutamate, guanylates and inosinates; natural flavoring materials such as hydrolyzates, cocoa, vanilla and caramel; essential oils and extracts such as anise oil, clove oil and the like and artificial flavoring materials such as vanillin, ethyl vanillin and the like. High intensity sweeteners such as aspartame and saccharin may also be used. Some of these flavoring materials may exist as solid particles.

Specific flavor adjuvants include but are not limited to the following: anise oil; ethyl-2-methyl butyrate; vanillin; cis-3-heptenol; cis-3-hexenol; trans-2-heptenal; butyl valerate; 2,3-diethyl pyrazine; methyl cyclo-pentenolone; benzaldehyde; valerian oil; 3,4- dimethoxy-phenol; amyl acetate; amyl cinnamate; y-butyryl lactone; furfural; trimethyl pyrazine; phenyl acetic acid; isovaleraldehyde; ethyl maltol; ethyl vanillin; ethyl valerate; ethyl butyrate; cocoa extract; coffee extract; peppermint oil; spearmint oil; clove oil; anethol; cardamom oil; wintergreen oil; cinnamic aldehyde; ethyl-2-methyl valerate; γ-hexenyl lactone; 2,4-decadienal; 2,4-heptadienal; methyl thiazole alcohol (4-methyl-5-β-hydroxyethyl thiazole); 2-methyl butanethiol; 4-mercapto-2-butanone; 3-mercapto-2-pentanone; 1-mercapto-2- propane; benzaldehyde; furfural; furfuryl alcohol; 2-mercapto propionic acid; alkyl pyrazine; methyl pyrazine; 2-ethyl-3-methyl pyrazine; tetramethyl pyrazine; polysulfides; dipropyl disulfide; methyl benzyl disulfide; alkyl thiophene; 2,3-dimethyl thiophene; 5-methyl furfural; acetyl furan; 2,4-decadienal; guiacol; phenyl acetaldehyde; β-decalactone; d-limonene; acetoin; amyl acetate; maltol; ethyl butyrate; levulinic acid; piperonal; ethyl acetate; n-octanal; n-pentanal; n-hexanal; diacetyl; monosodium glutamate; mono-potassium glutamate; sulfur-containing amino acids, e.g., cysteine; hydrolyzed vegetable protein; 2-methylfuran-3-thiol; 2-methyldihydrofuran-3-thiol; 2,5-dimethylfuran- 3-thiol; hydrolyzed fish protein; tetramethyl pyrazine; propylpropenyl disulfide; propylpropenyl trisulfide; diallyl disulfide; diallyl trisulfide; dipropenyl disulfide; dipropenyl trisulfide; 4-methyl-2-[(methyl-thio)-ethyl]-1,3-dithiolane; 4,5-dimethyl-2-(methylthiomethyl)-1,3-dithiolne; and 4-methyl-2-(methylthiomethyl)-1,3-dithiolane. These and other flavor ingredients are provided in U.S. Pat. Nos. 6,110,520 and 6,333,180.

It should be noted that flavor or fragrance materials and blends thereof will perform differently in the invention depending on their specific physical-chemical characteristics.

Examples of the appropriate therapeutic agents include hypnotics, sedatives, antiepileptics, awakening agents, psychoneurotropic agents, neuromuscular blocking agents, antispasmodic agents, antihistaminics, antiallergics, cardiotonics, antiarrhythmics, diuretics, hypotensives, vasopressors, antitussive expectorants, thyroid hormones, sexual hormones, antidiabetics, antitumor agents, antibiotics and chemotherapeutics, and narcotics.

Examples of diagnostic agents include, but are not limited to synthetic inorganic and organic compounds, proteins, peptides, polypeptides, polysaccharides and other sugars, lipids, and DNA and RNA nucleic acid sequences having diagnostic activities.

Examples of hydrophilic particles include hydrophilic silicas, silicates, dextrins, starches, minerals, sodium bicarbonate, acids, salts, clays, sugars, polyols, spray-dried flavors/fragrances, hydrocolloids, proteins, celluloses, flours of wheat, corn, potato, and rice, dried milk and dairy powders, spice, herb, and vegetable powders, meat powders, aqueous soluble polymers and combinations thereof. Virtually any finely divided hydrophilic particle may be used. Particles with a hydrophilic surface may be used regardless of the internal particle composition.

Smaller hydrophilic particles are preferred in creating products using the invention. Particles less than 500 microns may be used. But particles less than 50 microns are preferred, and particles less than 1 micron are most preferred. Nano sized particles, such as those used in the examples, function very well. This is probably due to the large available surface area available for interaction.

Page 7 of 20

Examples of liquid polyhydric materials are glycerine, propylene glycol, dipropylene glycol, aqueous solutions of sorbitol, isomalt, lactitol, and maltitol, an aqueous solution of a sugar, a starch, polyethylene glycol, an aqueous soluble polymer, an acid, a dextrin, and a combination thereof. An aqueous solution of virtually any material may be used.

Polymers that are soluble in the hydrophobic phase and thus promote a higher liquid viscosity are useful adjuncts to the product, creating more stable structures and improving performance. Virtually any polymer that is soluble in the hydrophobic phase may be used, but preferred combinations include:

Ethylcellulose in flavor/fragrance at levels of about 0.1 to about 20% by weight of the structured material;

Hydroxypropyl cellulose in flavor/fragrance at levels of about 0.1 to about 20% by weight of the structured material;

Ethylene vinyl acetate in flavor/fragrance at levels of about 0.1 to about 20% by weight of the structured material; and

Ethylcellulose in triglyceride oil at levels of about 0.1 to about 20% by weight of the structured material.

In addition, combinations of polymers are included. Additionally, hydrophobic phase materials may be combined in order to provide adequate solubilization of the selected polymer.

In addition to the foregoing components, various optional ingredients such as are conventionally used in the art, may be employed in the matrix of this invention. For example, colorants, pigments, hydrophobic particles, fillers, diluents, emulsifiers, preservatives, anti-oxidants, stabilizers, lubricants, and the like may be employed herein to enhance visual and/or functional characteristics.

The colorants of the present invention include, but are not limited to lakes, preparations containing lakes, oleoresins, pigments, and minerals. An example of a preparation containing lakes is Spectra Flecks™ (Sensient Technologies, St. Louis, Mo.).

Fillers include, for example, materials such as silicon dioxide, titanium dioxide, alumina, talc, kaolin, powdered cellulose and microcrystalline cellulose, as well as soluble materials such as mannitol, urea, sucrose, lactose, dextrose, sodium chloride and sorbitol. Suitable diluents include calcium phosphate, calcium sulfate, carboxymethylcellulose calcium, cellulose, cellulose acetate, dextrates, dextrin, dextrose, fructose, glyceryl palmitostearate, hydrogenated vegetable oil, kaolin, lactitol, lactose, magnesium carbonate, magnesium oxide, maltitol, maltodextrin, maltose, microcrystalline cellulose, polymethacrylates, powdered cellulose, pregelatinized starch, silicified microcrystalline cellulose, sodium chloride, sorbitol, starch, sucrose, sugar, talc, hydrogenated vegetable oil, and mixtures thereof. Emulsifiers include mono and di-glycerol esters of fatty acids, modified starch, polyglycerol esters, and sorbitol esters.

These structured products may be produced effectively via batch or continuous processes, as long as there is sufficient mixing to disperse the solids in the hydrophobic liquid and to contact the liquid polyhydric material with the solids. An extrusion process which provides both intimate mixing, product formation and the ability to post-treat particles is preferred when creating solid structures. A benefit of the invention is the fact that processing may take place at low temperatures, the only requirement being that the hydrophobic phase and the polyhydric material be liquids during the mixing.

The structured materials are useful for a variety of applications including:
Oral care applications, in which the structured materials can maintain discrete particles of flavor within an oral care product. Such oral care products include toothpaste, dental cream, gel or tooth powder, odontic, mouth washes, denture cleaning agents, pre- or post brushing rinse formulations, chewing gum, lozenges, candy and the like.
Baked products, in which the structured materials can help retain flavor during processing. Such baked products include bagels, biscuits, breads, cakes, cereal, cookies, crackers, cream puffs, doughnuts, empanadas, muffins, pancakes, pasta, pastries, pizza, sponge cakes and the like.
cleaning products, in which the structured materials can delay release of fragrance, thus promoting increased fragrance deposition. Such cleaning products include detergents, soap, shampoo, hair conditioners, dishwashing compositions, scrubbing compounds, window cleaners and the like.

In order to demonstrate the invention, the following examples were conducted. All U.S. patent and patent applications referenced herein are hereby incorporated by reference as if set forth in their entirety.

Unless noted to the contrary all weights are weight percent. Upon review of the foregoing, numerous adaptations, modifications and alterations will occur to the reviewer. These adaptations, modifications, and alterations will all be within the spirit of the invention. Accordingly, reference should be made to the appended claims in order to ascertain the scope of the present invention.

### EXAMPLE 1

The following formulation was processed via extrusion at ambient temperature:

| | % by weight |
|---|---|
| Flavor | 70.00 |
| Silicon Dioxide | 14.70 |
| Ethylcellulose Polymer | 4.00 |
| Glycerin | 11.29 |
| Color | 0.01 |

### EXAMPLE 2

Product from Example 1 was incorporated into a model toothpaste base. The particles survived the initial mixing process, retaining their visual identity in the base. When the toothpaste was evaluated, it was observed that the flavor onset from the particles was delayed several seconds compared to a control. Flavor from the particles continued to increase in intensity as brushing progressed. The particles were completely brushed out after 60 seconds. Flavor was still perceived 10 minutes post-brushing.

### EXAMPLE 3

To demonstrate the increase in viscosity possible when using the invention, the following experiment was performed. Peppermint flavor (containing Peppermint oil, menthol, and other essential oils, but no solvent) was mixed by hand with Aerosil 200 (fumed silicon dioxide). Glycerine (99% purity) was added as indicated. Viscosity was measured (shear rate sweep) using a Haake AR 2000 Rheometer at ambient temperature (about 25° C.).

| | Control | Sample 1 | Sample 2 | Sample 3 | Sample 4 |
|---|---|---|---|---|---|
| Peppermint | 14.00 | 14.00 | 14.00 | 14.00 | 14.00 |
| Flavor, grams | | | | | |
| Aerosil 200, | | 1.00 | 1.00 | 2.00 | 2.00 |
| grams | | | | | |
| Glycerine, | | | 0.72 | | 1.43 |
| grams | | | | | |
| Aerosil/ | | | 1.39 | | 1.40 |
| Glycerine ratio | | | | | |
| Comments | liquid | very | very soft | soft solid | semi- |
| | | thick | solid | | hard |
| | | liquid | | | solid |
| Viscosity | | | | | |
| (Pa · s) | | | | | |
| at specific | | | | | |
| shear rates | | | | | |
| 0.01/s | 0.058 | 54405 | 138700 | 479100 | 996250 |
| 0.02/s | 0.132 | 34730 | 79045 | 290200 | 618400 |
| 0.03/s | 0.083 | 21500 | 44965 | 177850 | 387250 |
| 0.04/s | 0.092 | 13300 | 27685 | 109900 | 245400 |
| 0.06/s | 0.065 | 8220 | 17100 | 67770 | 153750 |
| 0.10/s | 0.044 | 5073.5 | 10495 | 41720 | 82855 |

As can be seen, the addition of Aerosil increases the system viscosity by itself. Further addition of glycerine to the systems with dispersed Aerosil approximately doubles the viscosity, although the ratio of liquid to Aerosil increases significantly. This clearly shows the effect of the invention in creating structured systems.

### EXAMPLE 4

| | Sample 5 | Sample 6 | Sample 7 | Sample 8 |
|---|---|---|---|---|
| Peppermint Flavor, grams | 14.00 | 14.00 | | |
| Triglyceride, grams | | | 14.00 | 14.00 |
| Aerosil 200, grams | 0.25 | 0.25 | 0.50 | 0.50 |
| Glycerine, grams | | 1.50 | | 4.80 |
| Aerosil/Glycerine ratio | | 0.17 | | 0.10 |
| Comments | Thin liquid | Thin liquid | Very slightly | Thin liquid |
| | | phase with | thick liquid | phase with |
| | | loose gel | | loose gel |
| | | flocs | | flocs |
| Viscosity (Pa · s) at specific | | | | |
| shear rates | | | | |
| 0.01/s | 51.028 | 21395 | 208.84 | 3440.3 |
| 0.02/s | 23.21 | 7590.2 | 102.25 | 1998.6 |
| 0.03/s | 14.439 | 3258.4 | 66.587 | 1497.6 |
| 0.04/s | 11.019 | 2120.2 | 48.981 | 1297.2 |
| 0.06/s | 8.0961 | 1245.3 | 32.146 | 1042.8 |
| 0.10/s | 5.8207 | 680.88 | 19.176 | 791.22 |

As can be seen, addition of glycerine to the systems with dispersed Aerosil increases viscosity significantly. However, at low ratios of Aerosil to Glycerine such as 0.17 or 0.10, a structured monophasic or uniform product still can not be created.

### EXAMPLE 5

| | Sample 9 | Sample 10 | Sample 11 | Sample 12 | Sample 13 |
|---|---|---|---|---|---|
| Peppermint Flavor, % | 88.00 | 88.00 | 88.00 | 88.00 | 88.00 |
| Aerosil 200, % | 1.74 | 4.00 | 6.00 | 6.55 | 7.00 |
| Glycerine, % | 10.26 | 8.00 | 6.00 | 5.45 | 5.00 |
| Aerosil/Glycerine ratio | 0.17 | 0.50 | 1.00 | 1.20 | 1.40 |
| Comments | Not | Not | Homogeneous; | Uniform very | Uniform very |
| | homogeneous; | homogeneous; | smooth uniform | soft solid | soft solid |
| | thin liquid | thin liquid | soft gel | | |
| | phase with | phase with soft | | | |
| | floating gel | gel particles | | | |
| | flocs | | | | |
| Overnight soaking in | Not applicable | Not applicable | Does not hold | Swollen particle | Swollen particle |
| water (room temp) | for the test | for the test | shape; excess | but particle | but particle |
| | | | oil separation | identity | identity |
| | | | | maintained; | maintained; |
| | | | | slight oil | slight oil |
| | | | | separation | separation |
| Viscosity (Pa · s) at | 114 | 205 | 311 | 2315 | 2575 |
| 0.10/s shear rate | | | | | |
| | Sample 6 | Sample 14 | Sample 15 | Sample 16 | Sample 17 |
| Peppermint Flavor, % | 80.00 | 80.00 | 80.00 | 80.00 | 80.00 |
| Aerosil 200, % | 2.91 | 6.67 | 10.00 | 10.91 | 11.67 |
| Glycerine, % | 17.09 | 13.33 | 10.00 | 9.09 | 8.33 |
| Aerosil/Glycerine ratio | 0.17 | 0.5 | 1 | 1.2 | 1.4 |
| Comments | Not | Homogeneous; | Homogeneous; | Uniform semi- | Uniform semi- |
| | homogeneous; | thick | Paste-like gel, | hard solid | hard solid |
| | thin liquid | deformable gel | maintains shape | | |
| | phase with gel | | | | |
| | flocs | | | | |
| Overnight soaking in | Not applicable | Swollen but | Slightly | Slightly swollen | Slightly swollen |
| water (room temp) | for the test | particle identity | swollen but | but particle | but particle |
| | | maintained; | particle identity | identity | identity |
| | | moderate oil | maintained; | maintained; | maintained; |
| | | separation | slight oil | very slight oil | very slight oil |
| | | | separation | separation | separation |
| Viscosity (Pa · s) at | 681 | 6008 | 22118 | 27376 | 40156 |
| 0.10/s shear rate | | | | | |
| | Sample 18 | Sample 19 | Sample 20 | Sample 21 | Sample 22 |
| Peppermint Flavor, % | 70.00 | 70.00 | 70.00 | 70.00 | 70.00 |
| Aerosil 200, % | 4.36 | 10.00 | 15.00 | 16.36 | 17.50 |
| Glycerine, % | 25.64 | 20.00 | 15.00 | 13.64 | 12.50 |
| Aerosil/Glycerine ratio | 0.17 | 0.5 | 1 | 1.2 | 1.4 |
| Comments | Not | Homogeneous; | Homogeneous | Rigid gel; | Rigid gel; |
| | homogeneous; | stiff rigid gel, | rigid gel; | maintains | maintains |
| | gel flocs | slightly | maintains | shape | shape |
| | precipitate out | crumbly; | shape | | |
| | of solution and | maintains | | | |
| | settle on the | shape | | | |
| | bottom | | | | |
| Overnight soaking in | Not applicable | Slightly | Slightly | Slightly | Slightly |
| water (room temp) | for the test | swollen but | swollen but | swollen but | swollen but |
| | | particle | particle | particle | particle |
| | | identity | identity | identity | identity |
| | | maintained; | maintained; | maintained; | maintained; |
| | | moderate oil | slight oil | very slight oil | very slight oil |
| | | separation | separation | separation | separation |
| Viscosity (Pa · s) at | 565 | 20138 | 70725 | 75473 | 101680 |
| 0.10/s shear rate | | | | | |
| | Sample 23 | Sample 24 | Sample 25 | Sample 26 | Sample 27 |
| Peppermint Flavor, % | 50.00 | 50.00 | 50.00 | 50.00 | 50.00 |
| Aerosil 200, % | 7.26 | 16.67 | 25.00 | 27.27 | 29.17 |
| Glycerine, % | 42.74 | 33.33 | 25.00 | 22.73 | 20.83 |
| Aerosil/Glycerine ratio | 0.17 | 0.5 | 1 | 1.2 | 1.4 |
| Comments | Not | Expected to be | Expected to be | Expected to be | Expected to be |
| | homogeneous; | acceptable* | acceptable* | acceptable* | acceptable* |
| | thick, rigid gel | | | | |
| | completely | | | | |
| | separates from | | | | |
| | liquid phase | | | | |
| Overnight soaking in | Not applicable | Not tested | Not tested | Not tested | Not tested |
| water (room temp) | for the test | | | | |
| Viscosity (Pa · s) at | 37655 | | | | |
| 0.10/s shear rate | | | | | |

*Tests of these samples are considered not necessary as it is obvious that if the amounts of Aerosil and glycerine are increased with preferred ratios, more structured complex will be formed to allow a uniform dispersion the flavor.

### EXAMPLE 5 (CONTINUED)

As can be seen in Example 5, the present invention covers a range of possible compositions that result in from highly viscous fluid to fully solidified products. The products are homogeneous and contain a high level of active ingredient ranging from 50-90% by weight. In most cases, the active ingredient is a liquid such as a flavor or fragrance oil. To create a stable structure, the ratio of the silicon dioxide and the polyhydric alcohol must be carefully formulated. Acceptable formulations form a uniform or homogeneous viscous fluid or uniform semi-solid or solid products.

What is claimed is:

1 A structured material, comprising:
a hydrophilic particle;
a hydrophobic active ingredient; and
a polyhydric material,
wherein the active ingredient is 50-90% by weight of the structured material, and wherein the ratio of the hydrophilic particle to the polyhydric material is about 0.5 and greater.

2. The structured material of claim 1, wherein the hydrophilic particle is selected from the group consisting of a hydrophilic silica, a silicate, a dextrin, a starch, a mineral, a salt, an acid, sodium bicarbonate, a clay, a sugar, a polyol, a dried flavor/fragrance, a hydrocolloid, a protein, a cellulose, hydroxypropyl methyl cellulose, methyl cellulose, a wheat flour, corn, potato, rice, dried milk, a dairy powder, a spice, a herb, a vegetable powder, and a meat powder.

3. The structured material of claim 1, wherein the hydrophobic active ingredient is selected from the group consisting of a flavor, a fragrance, a pharmaceutical agent, a triglyceride oil, a triglyceride fat, a mineral oil, and a mixture thereof.

4. The structured material of claim 1, wherein the polyhydric material is selected from the group consisting of glycerine, propylene glycol, dipropylene glycol, an aqueous solution of sorbitol, an aqueous solution of isomalt, an aqueous solution of lactitol, an aqueous solution of maltitol, an aqueous solution of a sugar, a starch, an acid, a salt, a soluble polymer, and a dextrin.

5. The structured material of claim 1, wherein the hydrophilic particle is less than 500 microns.

6. The structured material of claim 1, wherein the hydrophilic particle is less than 50 microns.

7. The structured material of claim 1, wherein the hydrophilic particle is less than 1 micron.

8. The structured material of claim 1, wherein the hydrophilic particle is from about 5% to about 70% by weight of the structured material.

9. The structured material of claim 1, wherein the polyhydric material is from about 1% to about 20% by weight of the structured material.

10. The structured material of claim 1, further comprising a hydrophobically soluble polymer selected from the group consisting of hydroxypropyl cellulose, cellulose acetate butyrate, ethylene vinyl acetate, and ethylcellulose.

11. The structured material of claim 1, wherein the ratio of the hydrophilic particle to the polyhydric material is about 1.0 and greater.

12. The structured material of claim 1, wherein the ratio of the hydrophilic particle to the polyhydric material is about 1.2 and greater.

13. The structured material of claim 1, wherein the ratio of the hydrophilic particle to the polyhydric material is about 1.4 and greater.

14. A method for preparing a structured material, comprising:
providing a hydrophilic particle;
providing a hydrophobic active ingredient;
providing a polyhydric material; and
admixing said hydrophilic particle, said active ingredient, and said polyhydric material to form said structured material,
wherein the active ingredient is 50-90% by weight of the structured material, and wherein the ratio of the hydrophilic particle to the polyhydric material is about 0.5 and greater.

15. The method of claim 14, wherein the ratio of the hydrophilic particle to the polyhydric material is about 1.0 and greater.

16. The method of claim 14, wherein the ratio of the hydrophilic particle to the polyhydric material is about 1.2 and greater.

17. The method of claim 14, wherein the ratio of the hydrophilic particle to the polyhydric material is about 1.4 and greater.

18. A method for delivering a hydrophobic active ingredient to an ingestible composition comprising the steps of:
preparing the structured material of claim 1; and
providing said structured material into said ingestible composition.

19. A method for delivering an active ingredient to a cleaning composition comprising the steps of:
preparing the structured material of claim 1; and
providing said structured material into said cleaning composition.
